(19) United States
(12) Patent Application Publication
(10) Pub. No.: US 2023/0346505 A1
(43) Pub. Date: Nov. 2, 2023

(54) DEVICE FOR CARDIAC IMAGING AND EPICARDIAL ABLATION AND METHODS OF USE THEREOF

(71) Applicant: The Trustees of the University of Pennsylvania, Philadelphia, PA (US)

(72) Inventors: Timothy MARKMAN, Philadelphia, PA (US); Saman NAZARIAN, Bryn Mawr, PA (US)

(73) Assignee: The Trustees of the University of Pennsylvania, Philadelphia, PA (US)

(21) Appl. No.: 18/343,840

(22) Filed: Jun. 29, 2023

Related U.S. Application Data
(63) Continuation of application No. PCT/US2021/065688, filed on Dec. 30, 2021.
(60) Provisional application No. 63/133,616, filed on Jan. 4, 2021.

(51) Int. Cl. A61B 90/00; A61B 18/14

(52) U.S. Cl. CPC A61B 90/361; A61B 18/1492; A61B 2090/3614; A61B 2018/00363

(57) ABSTRACT

The present disclosure provides a device for imaging cardiac structures having at least a sheath, a wire-basket structure and an optical scope. The present disclosure also provides methods for imaging of epicardial space, epicardial ablation and cardiac ultrasound using the device disclosed herein.

### CROSS-REFERENCE TO RELATED APPLICATIONS

This application is a Continuation of International Patent Application No. Application No. PCT/US2021/065688, filed Dec. 30, 2021, which priority to U.S. Provisional Patent Application No. 63/133,616, filed Jan. 4, 2021, the contents of which are incorporated herein by reference in its entirety.

### BACKGROUND

The epicardial surface of the heart contains critical electrical connections or abnormal substrate that must frequently be targeted for successful elimination of cardiac arrhythmia. Endocardial catheter ablation can be used to target and eliminate arrhythmia circuitry from the interior surface of the heart. By some estimates, over 75,000 catheter ablations are performed each year in the U.S. for atrial fibrillation or ventricular tachycardia. However, endocardial catheter ablation procedures can be unsuccessful in arrhythmia elimination due to an inability to identify substrates deep to the endocardial surface and/or suboptimal endocardial lesion depth for targeting these substrates. Endocardial ablation is also associated with increased risk of thrombogenicity and associated risk of cerebrovascular accidents.

Epicardial catheter ablation can be performed by introducing a sheath into the pericardial space through a subxiphoid approach and passing an ablation catheter through the sheath. The procedure can be guided by fluoroscopy, which lacks soft tissue resolution and thus limits safety by potential injury to unseen bystanders including epicardial coronary arteries, phrenic nerve, esophagus, and lungs. With fluoroscopy, and without expansion of the pericardial space, ablation catheter apposition to pericardial rather than epicardial tissue is often unavoidable. This decreases ablation efficacy and increases the risk of damage to adjacent thoracic and mediastinal structures. Abnormal myocardial substrate is targeted by identifying abnormal, low voltage electrical signals. Unfortunately, these signals can also represent poor catheter contact with the epicardium or local epicardial fat, resulting in ineffective targeting and ablation delivery.

Given the proximity of ablation targets to epicardial coronary arteries, coronary angiography must be performed prior to and often during ablation to avoid coronary injury and iatrogenic acute myocardial infarction. This additional procedure, often requiring multiple fluoroscopy views and repeat acquisitions with extra contrast for subsequent ablations, is associated with increased cost, requiring the involvement of an interventional cardiologist, as well as the need for additional fluoroscopy, intravenous contrast, nephrotoxicity, and increased risk of coronary artery injury. Despite disappointing success rates for endocardial approaches for these arrhythmias, there is general reluctance to pursue epicardial ablation driven by concerns about safety and the ability to effectively target abnormal tissue. As such, there is a need in the field for a high-resolution imaging system within the epicardial space.

### SUMMARY

The purpose and advantages of the disclosed subject matter will be set forth in and are apparent from the description that follows, as well as will be learned by practice of the disclosed subject matter. Additional advantages of the disclosed subject matter will be realized and attained by the devices particularly pointed out in the written description and claims hereof, as well as from the appended drawings.

To achieve these and other advantages and in accordance with the purpose of the disclosed subject matter, as embodied and broadly described, the disclosed subject matter provides a device having a sheath, an optical scope positioned at the distal end of the sheath; and a wire-basket structure surrounding the optical scope, wherein the wire-basket structure has three or more wires having a first end and a second end, wherein the first end of each of the wires is attached near a distal end of the sheath and the second end of each of the wires is joined together at a point above the distal end of the sheath forming an ovoid shape. In certain embodiments, the optical scope includes a white-light camera and/or a near-infrared camera.

In certain embodiments, the device further includes a transducer coupled to the optical scope. In certain embodiments, the transducer includes one or more sensing electrodes located on the wire-basket structure.

In certain embodiments, the transducer is configured to transmit sound waves having a frequency ranging from about 3 MHz to about 71 MHz and receive echo signals.

In certain embodiments, the device of the present disclosure is configured to accept an ablation catheter.

In certain embodiments, the present disclosure is directed to a method including providing a device having a sheath an optical scope positioned at the distal end of the sheath, and a wire-basket structure surrounding the optical scope and one or more sensing electrodes positioned on the wire basket structure; inserting the device in an epicardial space of a subject; and acquiring images of a heart and associated structures. In certain embodiments, the method further includes measuring epicardial signals.

In certain embodiments, the method of the present disclosure further includes administering a fluorescent contrast agent to the subject; and imaging autonomic ganglia and/or scar on the epicardial surface of the heart of the subject with the optical scope, wherein the optical scope includes a near-infrared camera or a dual band white light and near-infrared camera system.

In certain embodiments, the method further includes inserting an ablation catheter through the sheath into the epicardial space of the subject. In certain embodiments, the method further includes performing targeted ablation of epicardial tissue. In certain other embodiments, the method includes performing ablation of autonomic ganglia and/or scar on the epicardial surface of the heart of the subject.

In certain alternative embodiments, the method further includes transmitting sound waves having a frequency ranging from about 3 MHz to about 71 MHz from the device; and receiving at the device one or more echo signals at the device.

In certain embodiments, the method further includes generating two-dimensional or three-dimensional images of the heart and the associated structures from the received echo signals.

In certain other embodiments, the method further includes determining one or more of speed and direction of blood flow within chambers of the heart, across valves, and/or in great vessels, using the received echo signals.

### BRIEF DESCRIPTION OF THE DRAWINGS

FIG. 1 shows a diagram illustrating exemplary components of a device according to some examples of the disclosed subject matter.

FIG. 2 shows a photograph of a device according to certain embodiments of the present disclosure.

FIG. 3 shows a device having a wire-basket structure according to certain embodiments of the present disclosure.

FIG. 4 provides a fluoroscopic view showing the device according to certain embodiments of the present disclosure.

FIG. 5A shows a still image from the device according to certain embodiments showing wire-basket structure separating epicardium from pericardium and epicardial coronary vessel.

FIG. 5B shows a still image from the device according to certain embodiments showing wire-basket structure separating epicardium from pericardium and left atrial appendage.

FIG. 6 shows a still image from the device according to certain embodiments showing wire-basket structure separating epicardium from pericardium and ablation catheter visualized in contact with epicardial tissue during ablation.

FIG. 7 shows a fluoroscopic view showing the device with the wire-basket structure according to certain embodiments pf the present disclosure. Ablation catheter is inserted through the sheath into the epicardial space.

FIG. 8 shows local epicardial signals (EPI) recorded from three bipolar electrodes on wire-basket structure. Recordings show signals before (left panel) and after (right panel) ablation with attenuation of bipolar signals.

### DETAILED DESCRIPTION

The present disclosure generally describes a a video-enabled white light and/or near infra-red (NIR) visualization scope onto a steerable epicardial sheath, which is also equipped with a wire tissue expander.

For clarity, but not by way of limitation, the detailed description of the presently disclosed subject matter is divided into the following subsections:
I. Definitions;
II. Device; and
III. Methods of Imaging and Ablation.

### I. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.”

The terms “comprise(s),” “include(s),” “having,” “has,” “can,” “contain(s),” and variants thereof, as used herein, are intended to be open-ended transitional phrases, terms or words that do not preclude additional acts or structures. The present disclosure also contemplates other embodiments “comprising,” “consisting of” and “consisting essentially of,” the embodiments or elements presented herein, whether explicitly set forth or not.

The term “about” or “approximately” means within an acceptable error range for the particular value as determined by one of ordinary skill in the art, which depends in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein the term “subject” refers to any animal (e.g., a mammal), including, but not limited to, humans, non-human primates, rodents, and the like, which is to be recipient of a particular treatment.

### II. Device

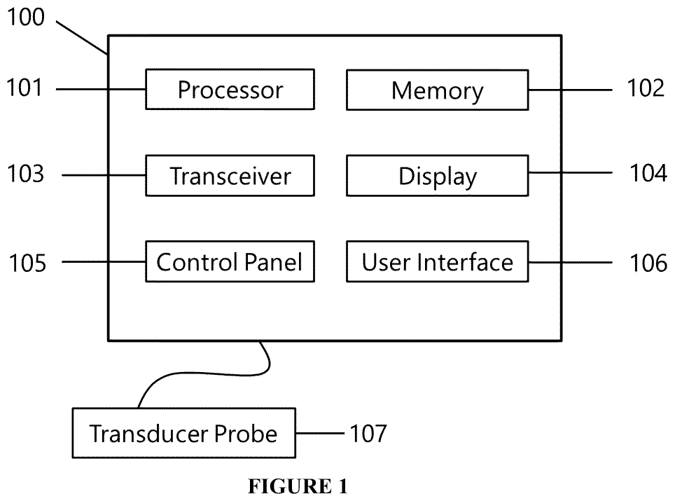

In certain embodiments, the present disclosure provides a device for imaging cardiac structures. FIG. 1 is an example of a device according to some non-limiting embodiments of the disclosed subject matter. FIG. 1 shows a device 100, such as a computer, mobile device, medical imaging device, ultrasound system or device, or any other device that includes a processor 101, memory 102, and/or graphical user interface 104. In certain embodiments, the device 100 is a visual/infrared endoscope. Each feature of FIG. 1 can be implemented by the device or a visual/infrared endoscope system, using various hardware, software, firmware, and/or one or more processors or circuitry, in connection with different embodiments of the disclosed subject matter.

In certain embodiments, the device can include at least one processor 101 or control unit. At least one memory 102 can also be provided in each device. Memory 102 can include computer program instructions or computer code contained therein, which instructions or code can be executed by the processor. The system can also include networked components communicating over a local network, a wide area network, wirelessly and/or wired, or by any other communication network or method of communication that allows communication of data from one system component to another.

In certain non-limiting embodiments, one or more transceivers 103 can be provided. The one or more transceivers 103 can receive signals from transducer probe 107, also referred to as transducer, which transmits and/or receives sound waves to and from the subject or body being examined. Transducer probe 107 can transmit the signal to apparatus 100 via a wireless or wired communication.

In some non-limiting embodiments, transducer probe 107 can be a single element or a multi-element transducer. In certain embodiments, the transducer 107 can include electrodes for electrophysiologic mapping. In certain embodiments, the electrodes can enable electrophysiologic mapping of the epicardial myocardium.

In certain embodiments, the transducer 107 is a piezoelectric transducer that can emit ultrasound sideward at an adjustable angle. In certain non-limiting embodiments, the transducer 107 can transmit sound waves of various frequencies and receive echo signals. The sound waves, for example, can range from a low bandwidth frequency of 3 Megahertz (MHz) to a high frequency of 71 MHz. Other non-limiting embodiments can use any other soundwave frequency. Higher frequencies can allow for the imaging of superficial structures, while lower frequencies can allow for the deeper tissue imaging with each typically providing different resolutions.

Transducer probe 107 can use either wired or wireless communication to send and/or receive information to apparatus 100. The transmitted information can be saved in memory 102, or in any other external memory or database.

In certain embodiments, the display 104 can be in a separate apparatus from device 100. In yet another example, instead of a display the apparatus can include a projector capable of projecting the image onto an external display or screen.

In certain non-limiting embodiments, at least one memory including computer program code can be configured to, when executed by the at least one processor, cause the apparatus to perform any or all of the processes described herein. Processor 101 can be embodied by any computational or data processing device, such as a central processing unit (CPU), digital signal processor (DSP), application specific integrated circuit (ASIC), programmable logic devices (PLDs), field programmable gate arrays (FPGAs), input/output (I/O) circuitry, digitally enhanced circuits, or comparable device, or any combination thereof. The processors can be implemented as a single controller, or a plurality of controllers or processors.

The device can also include a system control panel 105. System control panel 105 can include user interface 106. In some other embodiments user interface 106 can be a separate piece of hardware that is not located on control panel 105. User interface 106 can be a touch screen made of glass or any other material known to a person of skill in the art. In certain embodiments, user interface 106 can include an area with a molded indentation or a different texture, such as a sandblasted texture. The palm of the operator can be placed on the area of user interface 106 with a molded indentation or a different texture.

For firmware or software, the implementation can include modules or a unit of at least one chip set (for example, including procedures and/or functions). Memory 102 can independently be any suitable storage device, such as a non-transitory computer-readable medium, a hard disk drive (HDD), random access memory (RAM), flash memory, or other suitable memory. The memories can be combined on a single integrated circuit with a processor, or can be separate therefrom. Furthermore, the computer program instructions can be stored in the memory and be processed by the processors, and can be any suitable form of computer program code, for example, a compiled or interpreted computer program written in any suitable programming language. For example, in certain non-limiting embodiments, a non-transitory computer-readable medium can be encoded with computer instructions or one or more computer programs (such as added or updated software routine, applet or macro) that, when executed in hardware, can perform a process such as one of the processes described herein. Computer programs can be coded by a programming language, which can be a high-level programming language, such as objective-C, C, C++, C #, Java, etc., or a low-level programming language, such as a machine language, or assembler. Alternatively, certain non-limiting embodiments can be performed entirely in hardware.

In certain embodiments, the device of the present disclosure includes a camera. In certain embodiments, the camera can be used to enable visualization of tissues and structures. In certain embodiments, the device of the present disclosure can include a light source, such as but not limited to a white light source. In certain embodiments, the camera and the white light source enables visualization of abnormal tissues and structures, such as but not limited to those in epicardial space.

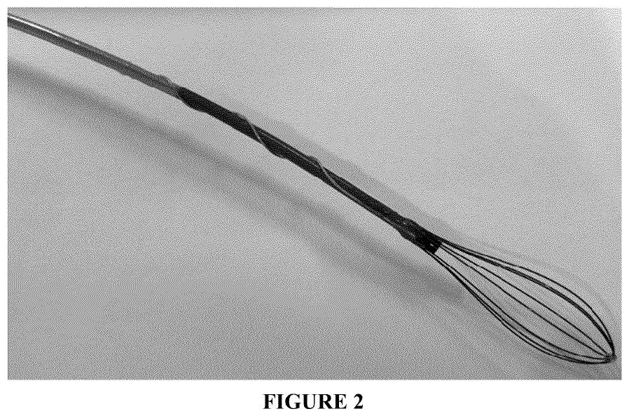

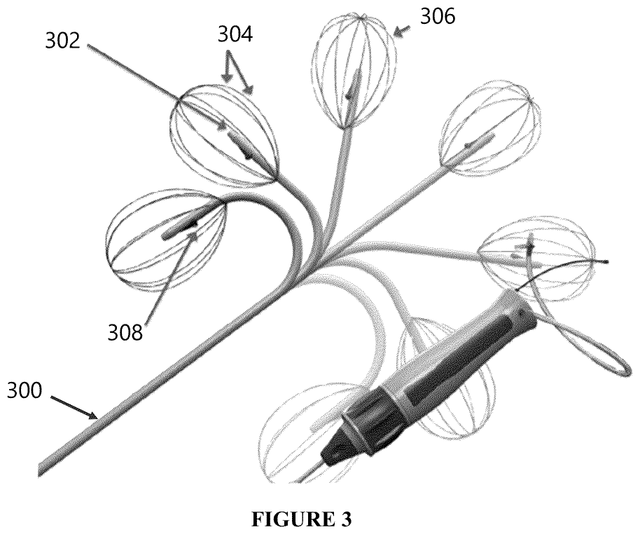

In certain embodiments, the device of the present disclosure can include a basket-like structure at one end of the device as shown in FIG. 2. FIG. 2 is a photograph of a device according to certain embodiments of the present disclosure. The basket-like structure is further illustrated in FIG. 3. As shown in FIG. 3, the device can include a sheath 300 and a sheath tip 302 that is surrounded by the wire-basket structure 306.

As shown, the wire-basket structure 306 can be a space expander, i.e., wire-basket structure 306 allows for to separation of tissue. As shown in FIG. 3, the wire-basket structure consists of four or more wires each having one end attached near the distal tip of the sheath and the other end being joined together at a point above the distal end of the sheath forming an ovoid shape with spaces between the wires. In certain embodiments the wire-basket structure has from 4 to 20 wires. In certain embodiments, the wire-basket structure 306 has a diameter from about 1 cm to about 3 cm or about 2 cm. In certain embodiments, the wire-basket structure has an expansion range of from about 0.5 mc to about 3.0 cm. In certain embodiments, each wire in the wire-basket structure 306 is about 4 cm long, but can range from about 1 cm to about 8 cm in length.

In certain embodiments, the wire-basket structure 306 allows separation of epicardial and pericardial tissue. In certain embodiments, the wire-basket structure 306 can be used to move pericardium and phrenic nerve. In certain embodiments, the wire-basket structure 306 can be used to move the esophagus away from the posterior left atrium and basal left ventricle.

In certain embodiments, the device of the present discloser can include electrodes 304, placed on the wire-basket structure 306. In certain embodiments, the electrodes can be used for electrophysiologic mapping. In certain embodiments, the electrodes 304 can provide electrophysiologic mapping of the epicardial myocardium. Electrophysical mapping can enable identification of abnormal, arrhythmogenic substrate within the tissue. As such, in certain embodiments the device of the present disclosure can include electrodes 304 for identification of abnormal, arrhythmogenic substrate within the cardiac tissue.

In certain embodiments, the sheath 300 can further include an optical scope 308 surrounded by the wire-basket structure 306. In certain embodiments, the optical scope can provide a view ranging from 180° to 360°. In certain particular embodiments the optical scope can provide a 180° view. In certain other embodiments, the optical scope can provide a 360° view. In certain embodiments, the optical scope can enable visualization of a catheter touching the electrodes 304 that are proximal and distal to the location of the scope. In certain embodiments, the optical scope is a fiber-optic camera. The fiber-optic camera can be located at the top of the sheath directed forward.

In certain embodiments, sheath 300 is steerable. In certain embodiments, the steerable sheath 300 cane used to navigate withing the pericardial space.

In certain embodiments, the device of the present disclosure can include a piezoelectric transducer the distal end emitting ultrasound sideward at an adjustable angle. In certain embodiments, the piezoelectric transducer can be used to perform echocardiography, such as but not limited to 2-D (two-dimensional) echocardiography, 3-D (three-dimensional) echocardiography, M-mode echocardiography, Doppler echocardiography and Color Doppler echocardiography.

In certain embodiments, the device of the present disclosure can further include a near-infrared camera. In certain embodiments, the optical scope is a high-definition, dual band (white light and near-infrared) camera system capable of emitting and detecting light in the near-infrared spectrum. In certain embodiments, the near-infrared camera can be used to visualize autonomic ganglia and scar on the epicardial surface of the heart. In such embodiments, a targeted fluorescent contrast agent is used to highlight autonomic ganglia and scar tissue. In certain non-limiting embodiments, the contrast agent is indocyanine green (ICG), an FDA approved fluorescent contrast agent that emits in the near-infrared spectrum (emission ˜805 nm). As known to a person of skill in the art, ICG is retained by scar with a longer contrast washout time compared to normal tissues, thereby enabling identification of regions of late uptake, which can represent potential arrhythmogenic substrate to target with ablation.

In certain embodiments, the sheath 300 can be configured to accept a catheter/device for performing epicardial procedures ranging from ablation to appendage closure, to biopsy, to injection, to delivery of pacing leads, to vascular interventions. In certain embodiments, the catheter is an ablation catheter. Size of the sheath is determined by the application, but can range from about 7 Fr to about 12 Fr inner diameter. The sheath outer diameter will range from 9 Fr to 28 Fr.

In certain embodiments, the device of the present disclosure can be used for epicardial tissue ablation. In certain embodiments, the device can be used to treat arrythmias, such as but not limited to ventricular tachycardia and atrial fibrillation. In certain embodiments, the device can be used in treatment of left atrial appendage occlusion. In certain embodiments, the device can be used for implantation of epicardial pacemaker leads.

### III. Methods of Imaging and Ablation

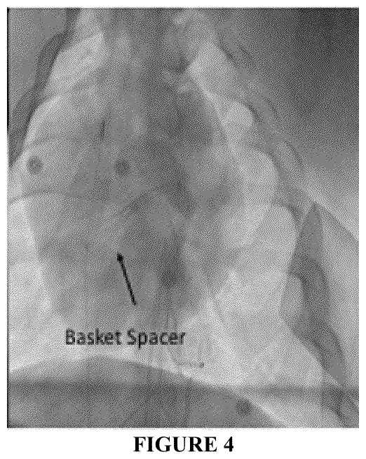

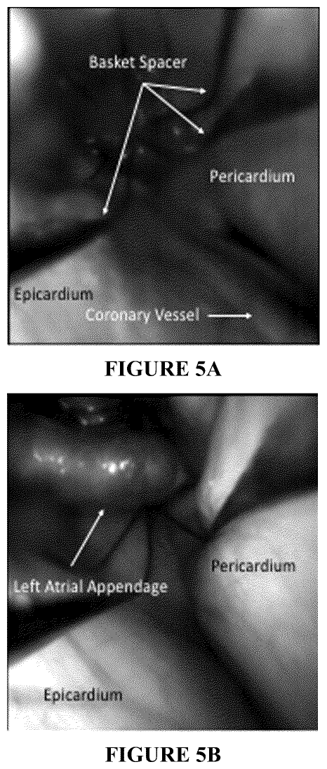

In certain embodiments, the present disclosure is directed to visualization of epicardial structures by guiding device including at least a sheath, an optical scope at a distal end of the sheath, which is surrounded by a wire-basket structure, into the epicardial space. In certain embodiments, the sheath is steerable, thus allowing navigation within the pericardial space. An example of this is shown in FIG. 4, which shows a fluoroscopic view of the sheath with the wire-basket structure at the distal end of the sheath placed within the epicardial space. In certain embodiments, the wire-basket structure provides separation of tissue. In certain embodiments, as shown in FIG. 5A, the wire-basket structure can separate epicardium from pericardium and epicardial coronary vessel. In certain embodiments, as shown in FIG. 5B, the wire-basket structure can separate epicardium from pericardium and left atrial appendage. Such separation of the tissue allows to achieve a better viewpoint of the cardiac structures and a clearer image can be obtained with the optical scope and thereby critical epicardial structures can be identified.

In certain embodiments, the method further includes performing electrophysiological measurements of the cardiac structures. In such embodiments, the wire-basket structure can include one or more sensing electrodes to measure epicardial signals. In certain embodiments, electrophysiological measurements can be used to identify abnormal epicardial signals.

In certain embodiments, the method further includes imaging of autonomic ganglia and scar on the epicardial surface of the heart. In such embodiments, the sheath can include a near-infrared camera. In certain embodiments, the sheath includes a dual band (white light and near-infrared) camera system capable of emitting and detecting light in the near-infrared spectrum. In certain embodiments, a subject is administered a fluorescent contrast agent to visually enhance the identification of autonomic ganglia and scar on the epicardial surface of the heart. In certain particular embodiments, the fluorescent contrast agent is indocyanine green. As known to a person of ordinary skill in the art, indocyanine green is retained by scar with a longer contrast washout time compared to normal tissues. As such, the regions of late uptake and enhancement of ICG representing potential arrhythmogenic substrate can be identified.

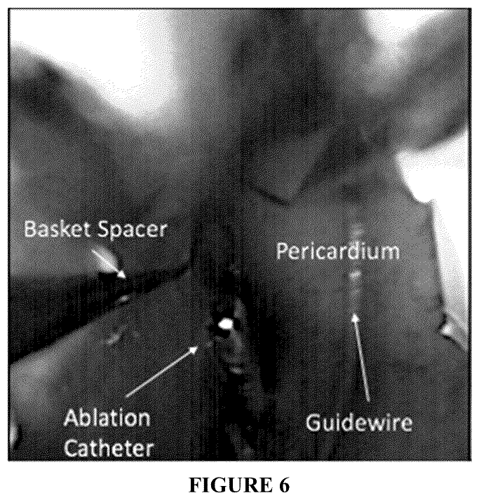

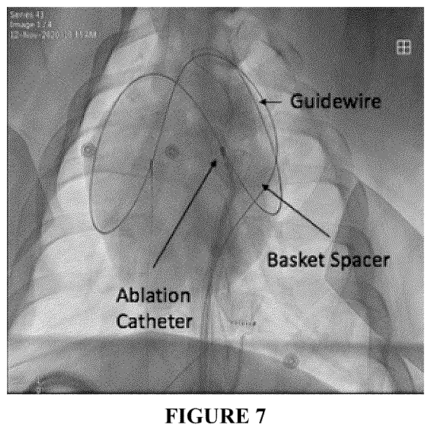

In certain embodiments, the present disclosure further provides methods of epicardial ablation. In certain embodiments, the method includes using a device including at least a sheath, an optical scope at a distal end of the sheath, which is surrounded by a wire-basket structure, as shown in FIG. 3. Size of the sheath is determined by the application, but can range from about 7 Fr to about 12 Fr inner diameter. The sheath outer diameter will range from 9 Fr to 28 Fr. In certain embodiments, an ablation catheter can be inserted though the sheath into the epicardial space, as shown in FIGS. 6 and 7.

As shown in FIG. 6, the wire-basket structure separates epicardium from pericardium and an ablation catheter is in direct contact with epicardial tissue during ablation. As seen, the use of the wire-basket structure enables reduction of collateral damage to adjacent structures, such as coronary injury and iatrogenic acute myocardial infarction. FIG. 7 provides a fluoroscopic view showing the sheath with a wire-basket structure at the distal end and the ablation catheter inserted through the sheath into the epicardial space.

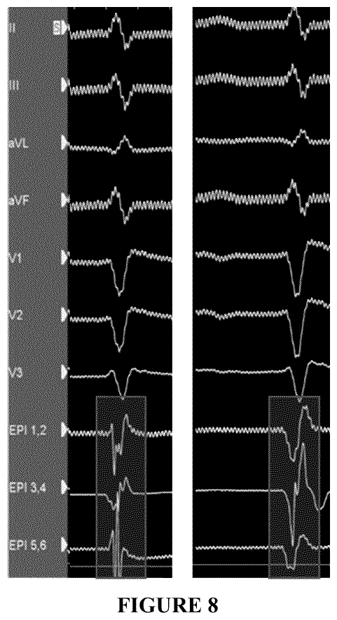

In certain embodiments, the wire-basket structure can include one or more sensing electrodes. In certain embodiments, abnormal epicardial signals can be identified with the electrodes and directly targeted for ablation. The identification of such signals is critical to localization of arrhythmia circuitry. For Example, FIG. 8 provides local epicardial signals (EPI) recorded from three bipolar electrodes on an arm of the basket expander. Recordings show signals before (left panel) and after (right panel) ablation with attenuation of bipolar signals.

In certain embodiments, the method can further include imaging of autonomic ganglia and scar on the epicardial surface of the heart. In such embodiments, the sheath can include a near-infrared camera. In certain embodiments, the sheath includes a dual band (white light and near-infrared) camera system capable of emitting and detecting light in the near-infrared spectrum. In certain embodiments, a subject is administered a fluorescent contrast agent to visually enhance the identification of autonomic ganglia and scar on the epicardial surface of the heart. In certain embodiments, the fluorescent contrast agent is indocyanine green. As known to a person of ordinary skill in the art, indocyanine green is retained by scar with a longer contrast washout time compared to normal tissues. As such, the regions of late uptake and enhancement of ICG representing potential arrhythmogenic substrate can be identified. In certain embodiments, the identified regions can be ablated by using an ablation catheter inserted through the sheath of the device of the present disclosure.

In certain embodiments, the present disclosure is directed to methods of performing echocardiography, by using a device as disclosed herein. The cardiac ultrasound includes but is not limited to 2-D (two-dimensional) echocardiography, 3-D (three-dimensional) echocardiography, M-mode echocardiography, Doppler echocardiography and Color Doppler echocardiography.

Although the presently disclosed subject matter and its advantages have been described in detail, it should be understood that various changes, substitutions and alterations can be made herein without departing from the spirit and scope of the disclosed subject matter. Moreover, the scope of the present application is not intended to be limited to the particular embodiments of the process, machine, manufacture, and composition of matter, methods and processes described in the specification.

As one of ordinary skill in the art will readily appreciate from the disclosed subject matter of the presently disclosed subject matter, processes, machines, manufacture, compositions of matter, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, or methods.

1. A device comprising:
a sheath;
an optical scope positioned at the distal end of the sheath; and
a wire-basket structure surrounding the optical scope, wherein the wire-basket structure comprises three or more wires having a first end and a second end, wherein the first end of each of the wires is attached near a distal end of the sheath and the second end of each of the wires is joined together at a point above the distal end of the sheath forming an ovoid shape.

2. The device of claim 1, wherein the optical scope comprises a white-light camera and/or a near-infrared camera.

3. The device of claim 1, wherein the device further comprises a transducer coupled to the optical scope.

4. The device of claim 3, wherein the transducer comprises one or more sensing electrodes located on the wire-basket structure.

5. The device of claim 3, wherein the transducer is configured to transmit sound waves having a frequency ranging from about 3 MHz to about 71 MHz and receive echo signals.

6. The device of claim 1, wherein the sheath is configured to have an inner lumen capable of accepting an ablation catheter or other epicardial tool.

7. A method comprising:
providing a device having a sheath an optical scope positioned at the distal end of the sheath, and a wire-basket structure surrounding the optical scope and one or more sensing electrodes positioned on the wire basket structure;
inserting the device in an epicardial space of a subject; and
acquiring images of a heart and associated structures.

8. The method of claim 7 further comprising measuring epicardial signals.

9. The method of claim 7, the method further comprising:
administering a fluorescent contrast agent to the subject; and
imaging autonomic ganglia and/or scar on the epicardial surface of the heart of the subject with the optical scope, wherein the optical scope comprises a near-infrared camera or a dual band white light and near-infrared camera system.

10. The method of claim 7, further comprising inserting an ablation catheter through the sheath into the epicardial space of the subject.

11. The method of claim 7, further comprising performing targeted ablation of epicardial tissue.

12. The method of claim 7, further comprising performing ablation of autonomic ganglia and/or scar on the epicardial surface of the heart of the subject.

13. The method of claim 7 further comprising:
transmitting sound waves having a frequency ranging from about 3 MHz to about 71 MHz from the device, and
receiving at the device one or more echo signals.

14. The method of claim 13, further comprising generating two-dimensional or three-dimensional images of the heart and the associated structures from the received echo signals.

15. The method of claim 13, further comprising determining one or more of speed and direction of blood flow within chambers of the heart, across valves, and/or in great vessels, using the received echo signals.
